(19) United States
(12) Patent Application Publication
(10) Pub. No.: US 2014/0039381 A1
(43) Pub. Date: Feb. 6, 2014

(54) MULTICHANNEL TROCAR

(71) Applicant: Michael M. Murr, Tampa, FL (US)

(72) Inventor: Michael M. Murr, Tampa, FL (US)

(73) Assignee: UNIVERSITY OF SOUTH FLORIDA, Tampa, FL (US)

(21) Appl. No.: 14/048,783

(22) Filed: Oct. 8, 2013

Related U.S. Application Data
(63) Continuation of application No. 12/824,796, filed on Jun. 28, 2010.
Application No. 12/824,796 is a continuation of application No. PCT/US2008/088418, filed on Dec. 29, 2008.
(60) Provisional application No. 61/016,948, filed on Dec. 27, 2007.

(51) Int. Cl. A61B 17/34

(52) U.S. Cl. CPC A61B 17/34; A61B 17/3474
USPC 604/26; 604/264

(57) ABSTRACT

A multi-channel trocar developed for use in minimal-access surgery/single incision laparoscopic surgery. The trocar permits surgical access of two or more articulating laparoscopic instruments or scopes through one skin incision using a dividing membrane to separate the working channels. This allows maximal use of robotic technology and permits more degrees of freedom at the operative site. The trocar is inserted through one single skin incision, in the usual manner. The trocar relocates the fulcrum and focal point of motion to the abdominal wall when using a set of interacting and interlocking diaphragms.

### CROSS REFERENCE TO RELATED APPLICATIONS

This application is a continuation of and claims priority to currently pending U.S. patent application Ser. No. 12/824,796, entitled “Multichannel Trocar”, filed Jun. 28, 2010, which is a continuation of and claims priority to prior filed International Application, Serial Number PCT/US2008/088418, filed Dec. 29, 2008, which claims priority to U.S. Provisional Patent Application No. 61/016,948, filed Dec. 27, 2007, each of which is hereby incorporated by reference into this disclosure.

### FIELD OF INVENTION

This invention relates to surgical trocars. More specifically, the invention is a surgical trocar with multiple channels for use in performing minimally invasive surgery.

### BACKGROUND OF THE INVENTION

Laparoscopic surgical techniques have been developed in order to avoid large skin incisions associated with traditional surgery. The abdomen or surgical space is inflated to enlarge the cavity and allow for the surgical procedure, and a small incision provides an access port for an endoscope and surgical instruments. These minimally invasive surgical procedures involve percutaneously accessing an internal surgical site with small-diameter trocars. Various instruments used in the procedure are inserted, previously one at a time, through the working channel of the trocar to perform the surgery. In order to maintain the insufflation pressure when the instrument is inserted through the trocar, a valve is provided in the housing to form a seal around the instrument.

Traditional laparoscopic surgeries require multiple trocars be inserted into a patient, in multiple incision sites. Surgeons use multiple trocars to channel laparoscopic instruments into the abdomen and position them in a triangulation with respect to the area of interest. The trocars penetrate the skin via an obturator and permit access to the desired surgical site. Typical surgical trocars include a cannula and a valve housing that define a working channel, for example extending through an abdominal wall and into a body cavity. A viewing scope is introduced through one trocar, and instruments are introduced through other appropriately placed trocars while viewing the operative site on a video monitor connected to the viewing scope. Such set up allows a surgeon to perform a wide variety of surgical procedures requiring only several 5 to 12 mm punctures at the surgical site. Consequently, patient trauma and recovery time are greatly reduced. Minimally invasive surgical procedures include laparoscopic procedures which involve the insufflation of the patient's abdominal region to raise the abdominal wall and create sufficient operating space to perform a desired procedure. Typically, an insufflation needle is utilized to insufflate the abdominal region.

However, the use of multiple trocars increases the likelihood of organ injury during the insertion of the trocar. Major problem with laproscopic surgeries is the risk of internal damage from insertion of the trocar. The FDA estimates that between 3 and 6% of minimally invasive surgical complications are a result of trocar-associated injury.

Minimally invasive surgery has also evolved into single port access (SPA) surgery, wherein a single incision between about 1 cm and about 5 cm is used for intraperitoneal access. Generally, the surgeon performs an incision at the umbilicus and one or two trocars are used by the surgeon (active trocars), while one or two additional trocars are utilized by a surgical assistant for organ and/or tissue retraction (passive trocars). For example, laparoscopic cholecystectomy (gallbladder removal) is generally performed using three trocars placed in the umbilicus (belly button); one for a telescope and two active trocars for surgical instrumentation. As a result of this procedure, the gall bladder is extracted through the umbilicus, with the subsequent scar not visible.

Introduction of ‘robotic’ technology has enabled surgical device companies to design laparoscopic instruments that have 180-360 degrees of rotation/articulation, thereby eliminating the need to use multiple trocars through multiple incisions. Currently techniques use one skin incision but multiple small trocars; resulting in crowding and hindered repetitive motion.

### SUMMARY OF THE INVENTION

The present invention takes advantage of current technologies, such as robotic surgical devices. These current technologies are increasingly used for single port access surgery. The majority of these surgeries use two or three trocars in a limited space of about 2 to 5 cm. However, current trocars are bulky, limiting the usefulness of current endoscopic techniques. As such, a multichannel trocar is disclosed for use with current endoscopic procedures. The trocar consists of an assembly body with a proximal end and a distal end with a gasket set disposed on the proximal end of the assembly body. The trocar uses a multichannel divider in the proximal end of the assembly body, running from the gasket set and parallel to the longitudinal axis of the assembly body, to divide the assembly body's peritoneal space into two or more working channels. The multichannel divider may run the entire length of the assembly body or just a portion thereof. In some embodiments, the trocar also has a series of finger holds on the outer surface of the assembly body to allow manipulation of the trocar.

The trocar may be constructed of any biocompatible material. Exemplary materials include stainless steel, surgical steel, titanium alloy, and thermoplastic. The biocompatible material may be further coated in a hydrophilic coating, such as polyvinylpyrrolidone, polyurethane, and polyvinybutyrol. The assembly body of the trocar may be comprised of two separable units, a proximal assembly body and a distal assembly body. In these embodiments, the proximal assembly body is adapted to fit into the proximal edge of distal assembly body and form an air-tight seal. In specific embodiments, the proximal assembly body can be rotated to adjust the orientation of the trocars from anterior-posterior to lateral-medial orientation.

A gas port may be disposed in the assembly body, such that the gas port may be connected to a gas supply for insufflating a patient's body cavity for surgery. In specific embodiments, a gas port valve is disposed on the outer surface of the assembly body and controls flow of gas through the gas port.

The trocar also may contain an instrument seal disposed in the assembly body's peritoneal space, such as a diaphragm, an air seal, a septum, a flapper seal, a braid, and a duckbill valve. The instrument seal may be constructed of a compound capable of forming an air-tight seal, such as polyester, para-phenylenediamine and terephthaloyl chloride polymer, carbon fiber, expanded PTFE, meta-phenylenediamine and terephthaloyl chloride polymer, nylon, fiber glass, cotton, polypropylene and ceramic, rubber, latex, silicone, polyurethane, polyisoprene, polystyrene and polybutadiene polymer, urethane, polyethylene, polyisoprene, polyvinylchloride, ethylene propylene diene monomer, neoprene, and styrene butadiene. In specific embodiments, instrument seal is also coated in at least one additional compound. Useful compounds are hydrophilic polymer coatings, Teflon, thermoplastic, cyanoacrylate, parylene, plasma surface treatments, cornstarch powder, silicone oil, silicone grease, astroglide lubricants, mineral oil, glycerin, alcohol, saline, Teflon lubricants, Krytox lubricants, molybdenum disulfide lubricants, and graphite. Where the instrument seal is a diaphragm, the diaphragm may be interacting and interlocking Alternatively, the instrument seal is an air seal, which comprises a pump connected to the assembly body's peritoneal space to allow transfer of gas from the pump to the assembly body's peritoneal space. The pump transfers a continuous supply of gas to at least one pressure barrier output jet, thereby forming a pressure barrier with the gas expelled by the at least one pressure barrier output jet. Air pressure differentials result in a seal, preventing flow of air from a surgical room to the patient's body cavity.

### BRIEF DESCRIPTION OF THE DRAWINGS

For a fuller understanding of the invention, reference should be made to the following detailed description, taken in connection with the accompanying drawings, in which:

FIG. 1 is a proximal side view of the assembled trocar of the present invention with two working channels. As shown in the figure, a gas supply valve is disposed on the outer surface of the assembly body.

FIG. 2 is a distal side view of the assembled trocar of the present invention. As shown in the figure, a gas supply valve is disposed on the outer surface of the assembly body.

FIG. 3 is a proximal view of the assembled trocar of the present invention with two working channels.

FIG. 4 is an illustration showing the orientation adjustment of the trocar from an anterior-posterior to lateral-medial orientation.

FIG. 5 is an illustration of the components of the trocar of the present invention using a diaphragm instrument seal.

FIG. 6 is a proximal view of the components of the trocar of the present invention using a diaphragm instrument seal.

FIG. 7 is a proximal side view of the components of the trocar of the present invention using a diaphragm instrument seal.

FIG. 8 is an illustration of the components of the trocar of the present invention using an air pressure instrument seal

### DETAILED DESCRIPTION OF THE PREFERRED EMBODIMENT

This ‘multi-channel trocar’ allows for introduction of two or more articulating laparoscopic instruments or scopes through one skin incision; one channel could be used to introduce a hard-tip or flexible tip laparoscope. This trocar capitalizes on robotic technology that allows more degrees of freedom at the operative site. A single skin incision is introduced, for example in the umbilicus of a patient, and the trocar is inserted through the peritoneum. The trocar is then positioned in direct trajectory with respect to the operative site.

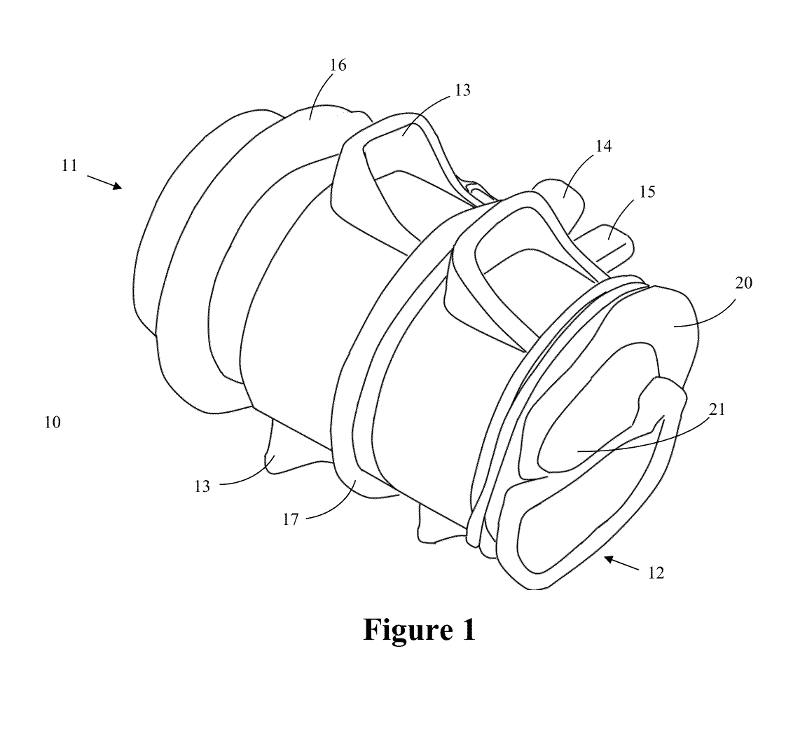

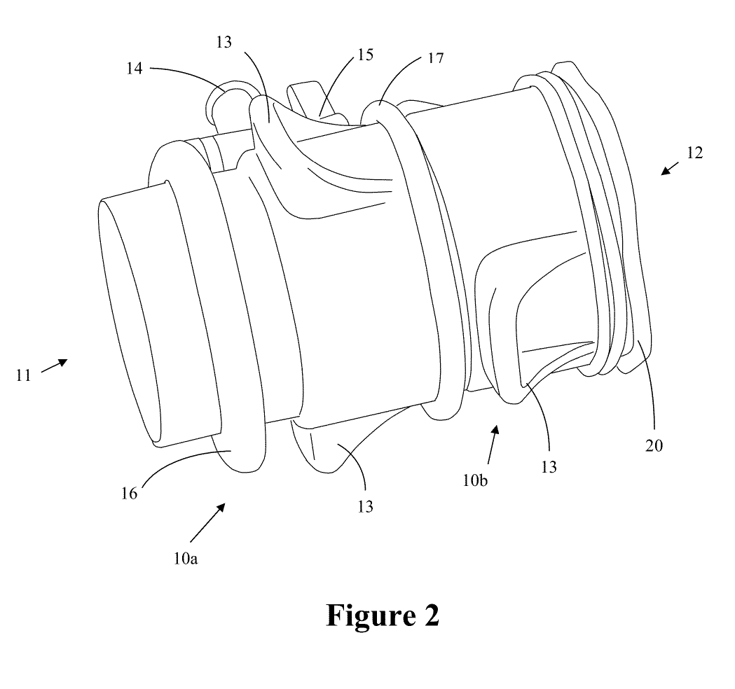

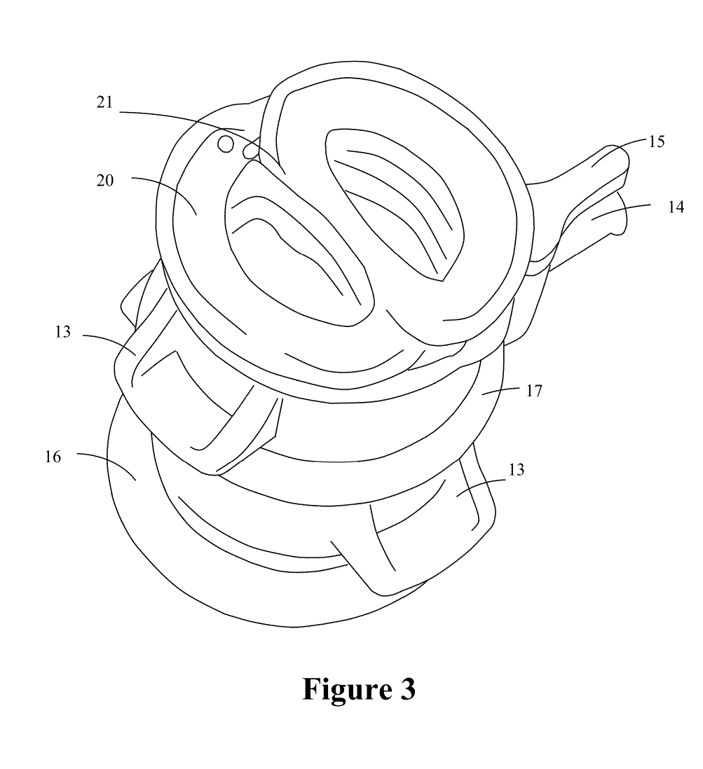

Disclosed is a trocar that provides distinct channels within the multichannel assembly, permitting multiple medical instruments to be introduced into the body cavity simultaneously. The trocar includes multichannel assembly body 10 with distal end 11 and proximal end 12, seen in FIGS. 1 and 2. A plurality of finger holds 13 are disposed along assembly body 10 to allow manipulation of the multichannel assembly body. Gasket set 20 is disposed on the proximal end of assembly body 10, allows the trocar to create a gas-tight seal. In referring to the opposite ends of the trocar, the “proximal end” refers to that part of the trocar which is closest to the operator or physician endoscopist (hereinafter collectively referred to as “operator”) performing the procedure, and the “distal end” refers to that part of the scope or guide farthest from the operator or physician endoscopist. Gasket set 20 includes multichannel divider 21, which runs parallel to the longitudinal axis of assembly body 10 and divides the internal space of multichannel assembly body 10 into a plurality of parallel channels, as seen in FIG. 3, 5. Multichannel divider 21 may run the entire length of multichannel assembly body 10, or a portion thereof.

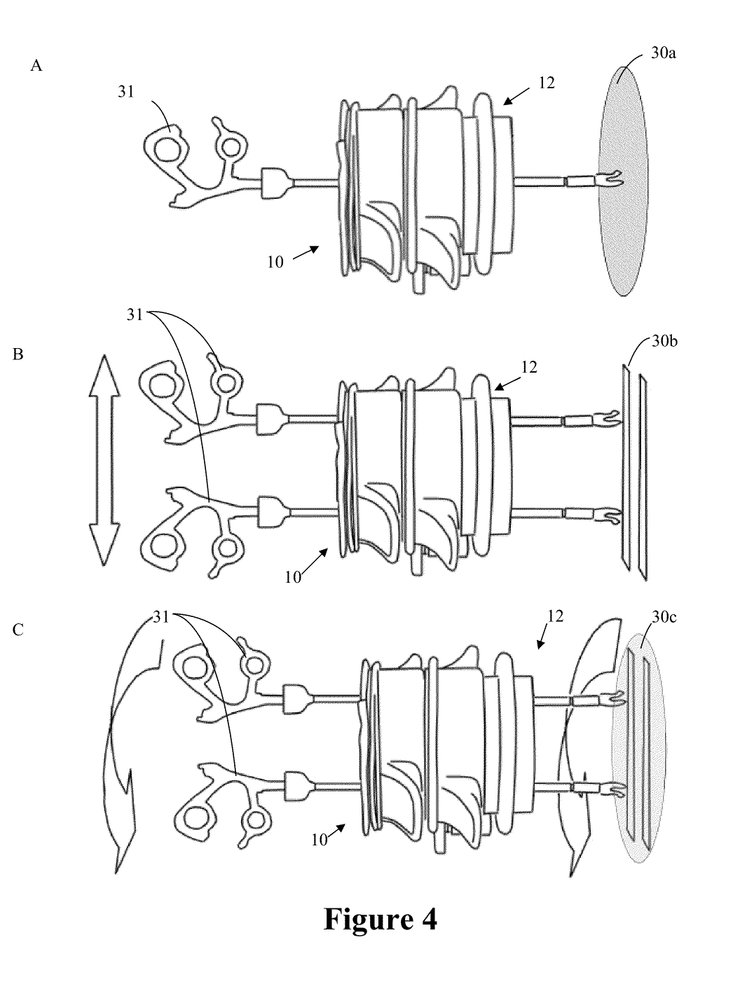

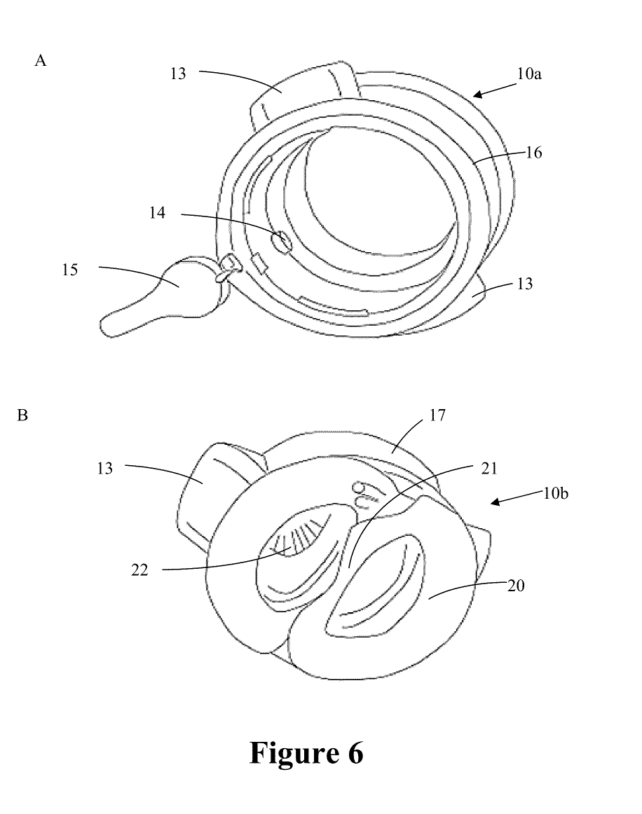

Distal to gasket set 20 on assembly body 10 is gas port 14 which can be connected to a gas supply, not shown, thereby providing a gas, such as carbon dioxide, into a patient's body cavity to create or maintain pneumoperitoneum. Also disposed on multichannel assembly body 10, proximal and adjacent to gas port 14, is gas supply valve 15, seen in FIG. 2, which controls the amount of gas that flows through gas port 14. Distal seal 16 is disposed on the distal head of assembly body 10 and is adapted to provide an air-tight seal with a patient's body cavity. Assembly body 10 is composed of distal assembly body 10a and proximal assembly body 10b, as seen in FIGS. 2, 5-7. Assembly body seal 17 is disposed about the middle of assembly body 10, providing a seal for distal assembly body 10a and proximal assembly body 10b. The trocar may be used as a single-instrument entry point, depicted in FIG. 4(a). In this embodiment, the instrument is provided full rotation access 30a. The trocar also permits use of multiple instruments 31, as seen in FIG. 4(b). However, in this embodiment, each instrument is limited in its range of motion 30b. The proximal portion of the trocar can be dialed in a clock-wise manner to change position of the trocars with respect to the operative site, and expanding the range of motion 30c of instruments 31. This permits the user to adjust the anterior-posterior to lateral-medial orientation, as seen in FIG. 4(c), and thereby allowing use of currently available fixed tips instruments.

Assembly body 10 is constructed of a durable, biocompatible material such as stainless steel, titanium alloy, or thermoplastic capable of withstanding repeated high temperature cleaning and sterilization. In some embodiments, the assembly body is coated in a hydrophilic coating. Appropriate hydrophilic coatings would include polyvinylpyrrolidone, polyurethane, or polyvinylbutyrol polymers. Appropriate molding compounds, which could alternatively also be applied as coatings, include hydrophilic polymer blends with thermoplastic polyurethane or polyvinylbutyrol and hydrophilic polyvinylpyrrolidone or other poly(N-vinyl lactans). An appropriate hydrophilic coating will reduce the coefficient of friction for stainless steel and can reduce the coefficients of friction for plastics.

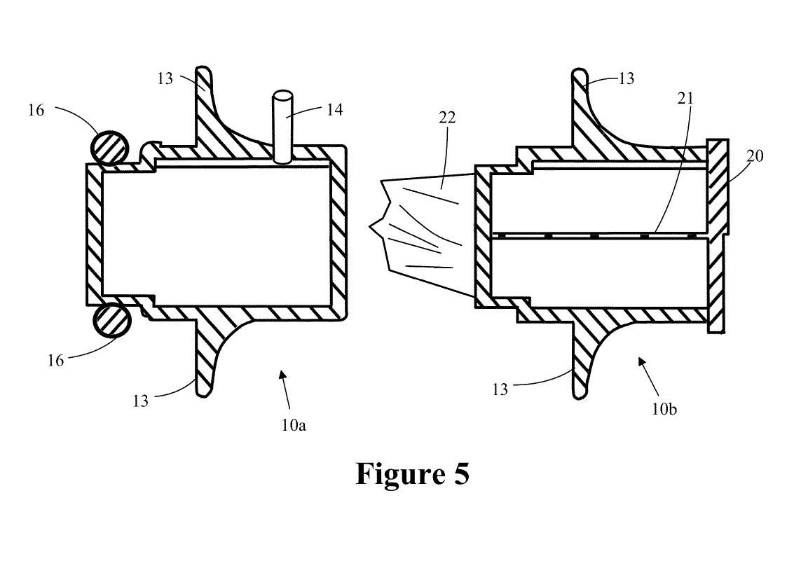

Assembly body 10 utilizes instrument seals 22 in the inner cavity of assembly body 10, seen in FIGS. 5-7, thereby allowing a surgeon to insufflate the patient's body cavity with gas. Surgical instruments vary in size and diameter, typically between about 3.5 mm to about 12.9 mm. In typical trocars, when an instrument is required that possesses a diameter outside the gas seal range, the entire trocar or valve housing needed replacement with a larger valve that could accommodate the new instrument. The instrument seals of the present trocar are constructed to prevent this replacement. Thus, the instrument seals may be interlocking diaphragms, an air seal, septum, valve, braid, and duckbill seal, such as those discussed below.

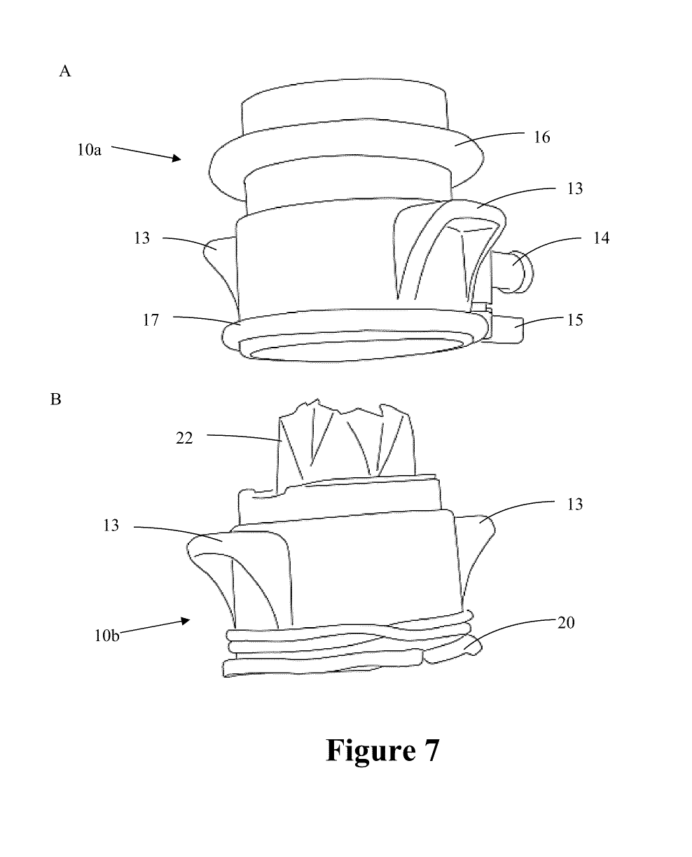

Instrument seals 22 may be a set of interacting and interlocking diaphragms in some embodiments. The diaphragms are disposed in proximal assembly body 10b and attached to the distal end of multichannel divider 21, as seen in FIGS. 5 and 7. As an endoscopic instrument is inserted into one of the plurality of channels formed by multichannel divider 21, the endoscopic instrument pushes through the diaphragms, which close around the endoscopic instrument, sealing the channel and preventing escape of pneumoperitoneal gases. The diaphragms are constructed of elastic materials that adjust to tightly fit around a surgical instrument, thereby preventing escape of insufflation gases. Moreover, the interacting and interlocking diaphragms relocate the fulcrum and focal point of motion for the endoscopic instruments to the abdominal wall. The diaphragms extend into the inner cavity of distal assembly body 10a, sealing at about where the distal end of the trocar contacts the body cavity. When an endoscopic instrument is sealed by the diaphragms, the pressure exerted by the diaphragms results in the aforementioned fulcrum relocation. The diaphragm may be coated or treated with a variety of materials to reduce friction between the inserted instruments and the gel material. Examples include hydrophilic polymer coatings, Teflon (PTFE) coatings, thermoplastic coatings, cyanoacrylate coatings, Parylene coatings, plasma surface treatments, cornstarch powder coatings and lubricants. Examples of useful lubricants include silicone oil, silicone grease, Astroglide lubricants, mineral oil, glycerin, alcohol, saline, Teflon (PTFE) lubricants, Krytox lubricants, molybdenum disulfide lubricants and graphite.

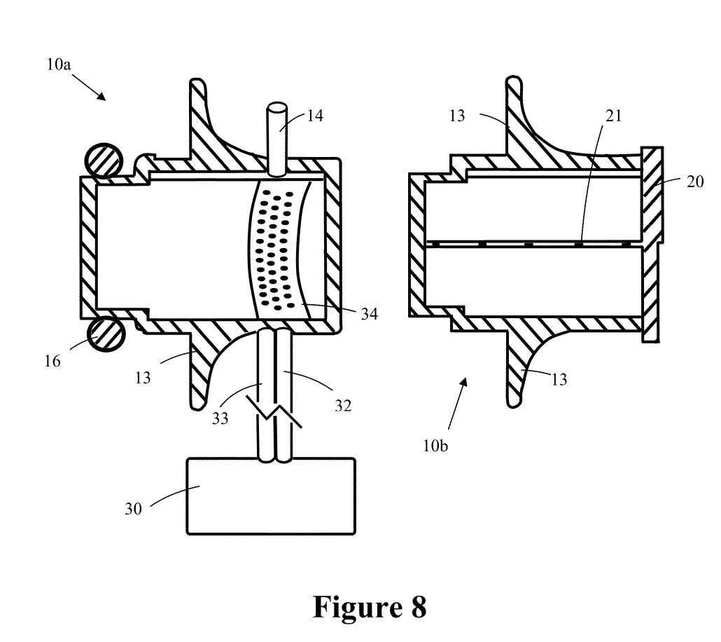

In other embodiments, the instrument seals 22 may be an air seal, which uses a pressure barrier to maintain insufflation of the intra-abdominal space. Pump 30, seen in FIG. 8, collects gas from the intra-abdominal space via gas recirculation input tube 32 and recirculates the gas via recirculation output tube 33 to create to a pressure barrier using pressure barrier output jets 34. The air pressure formed from the output jets of the pressure barrier is sufficiently powerful enough to maintain pneumoperitoneum during endoscopic surgery. This allows the surgeon to insert surgical instruments into the trocar without need to consider the diameter of the instruments.

The trocar may alternatively use a septum valve, such as universal seal septum valves, to accommodate different ranges of instrument diameters. These universal seals are typically of elastic material and may also utilize multiple septum seals to accommodate instruments having various diameters. For example, a septum valve may include one septum seal to engage large diameter instruments and another septum seal to engage smaller diameter instruments. The septum valve must perform when a sharp instrument is inserted off-center or when an instrument is moved radially after insertion and should allow the insertion and removal of instruments including tissue removal. The septum seal may be configured to float within assembly body 10 to minimize the cat-eye effect around the inserted instrument, which can result in seal leakage during manipulation of the instrument. The septum seal may be molded from a gel material possessing a low durometer that enables it to extrude through interstitial spaces of assembly body 10. Exemplary materials are composite materials comprising mineral oil and a thermoplastic elastomer such as a Kraton material. Alternatively, the septum seal may be manufactured from a closed cell foam material or an open cell foam material sealed with a film coating. Examples of the foamed materials include silicone, urethane, Kraton, polyethylene, polyisoprene, polyvinylchloride (PVC), polyurethane, ethylene propylene diene monomer (EPDM), neoprene and styrene butadiene (SBR). The septum seals may be coated or treated with a variety of materials and/or processes designed to reduce friction between the inserted instruments and the gel material. Examples include hydrophilic polymer coatings, Teflon (PTFE) coatings, thermoplastic coatings, cyanoacrylate coatings, Parylene coatings, plasma surface treatments, cornstarch powder coatings and chlorination treatments. The septum seals may also be lubricated with a variety of materials to facilitate the insertion and withdrawal of instruments. Examples of these materials include silicone oil, silicone grease, liquid soaps, Astroglide lubricants, mineral oil, glycerin, alcohol, saline, Teflon (PTFE) lubricants, Krytox lubricants, molybdenum disulfide lubricants and graphite.

Instrument seals 22 may also include a valve housing. The valve housing includes an access port, which comprises a braid or mesh tube having an aperture or central sealing orifice adapted to receive a wide range of instrument sizes. The braid is made of natural and synthetic monofilament thread materials including polyester, Kevlar, carbon fiber, Gore-Tex (expanded PTFE), Nomex, nylon, fiber glass, cotton, polypropylene and ceramic, which provides a low-friction, expandable lead-in to aperture, allowing the braid to engage and seal endoscopic instruments having diameters ranging from about 3.5 mm to about 12.9 mm. The braid is generally shaped like an hourglass having converging and diverging sidewalls that facilitate the insertion and removal of instruments through access port. The braid may be permanently coated or treated with a variety of materials and/or processes designed to reduce friction between inserted instruments and the braid, including any soft or low-durometer elastomeric material. The elastomeric material could be at least one of a thermoplastic and a thermoset. Examples of the elastomeric materials include silicone, polyurethane, polyisoprene and Kraton. Examples of other coatings and treatments include hydrophilic polymer coatings, Teflon (PTFE) coatings, cyanoacrylate coatings, Parylene coatings, plasma surface treatments and chlorination treatments.

Instrument seals 22 may further comprise a double duckbill valve, which maintains pneumoperitoneum in the absence of inserted instrumentation as described in the incorporated U.S. Pat. No. 6,162,196. When an instrument is present in a channel, the forms a seal with the instrument in order to seal the channel.

The trocar is useful in performing endoscopic surgeries, such as single port access (SPA) surgeries. In SPA surgery, a small incision is made in the surface of the skin or epidermis of the body cavity wall of a patient. The incision is about 2 cm to about 5 cm in length. The surgeon may then insert a finger to pry away subcutaneous fascia and other anatomical connection to the epidermis. The trocar is then inserted into the incision. Manual force is applied to the proximal face of the assembly housing, with or without use of a Kelly clamp, the trocar is pushed into the body cavity wall, such that distal seal 16 is seated within the body cavity, beneath the fascia, and sealed to the fascia. Instrument seals 22 are confirmed closed or closed by initiating pump 30 for the air seal. Gas supply is opened allowing gas to enter gas port 14. Gas flow is controlled by gas supply valve 15, permitting the surgeon to adjust the level of pneumoperitoneum during endoscopic surgery.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of an endoscopic trocar, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween.

What is claimed is:

1. A trocar, comprising:
an assembly body further comprising
a distal assembly body having a distal end and a proximal end with an interstitial space disposed therebetween;
a proximal assembly body having a distal end and a proximal end with an interstitial space disposed therebetween, wherein the distal end of the proximal assembly body is rotatably disposed on the proximal end of the distal assembly body;
an assembly body seal disposed on the proximal end of the distal assembly body wherein the assembly body seal is adapted to sealingly engage the distal end of the proximal assembly body; and
wherein the distal assembly body and the proximal assembly body are rotatably engaged;
a gasket set disposed on the proximal end of the proximal assembly body; and
a multichannel divider disposed in the proximal assembly body, extending longitudinally through the interstitial space in the proximal assembly body, thereby dividing at least a portion of the interstitial space of the proximal assembly body into two or more channels.

2. The trocar of claim 1, further comprising a plurality of finger holds disposed on the outer surface of the assembly body.

3. The trocar of claim 1, further comprising a gas port disposed in the distal assembly body, in fluid connection to the interstitial space of the distal assembly body.

4. The trocar of claim 3, further comprising a gas supply valve disposed on the outer surface of the assembly body, wherein the valve controls flow of gas through the gas port.

5. The trocar of claim 1, wherein the assembly body comprises at least one composition selected from the group consisting of stainless steel, surgical steel, titanium alloy, and thermoplastic.

6. The trocar of claim 5, wherein the at least one composition is coated in a hydrophilic coating selected from the group consisting of polyvinylpyrrolidone, polyurethane, and polyvinylbutyrol.

7. The trocar of claim 1, further comprising an instrument seal disposed on the distal end of the proximal assembly body.

8. The trocar of claim 7, wherein the instrument seal comprises at least one compound selected from the group consisting of polyester, para-phenylenediamine and terephthaloyl chloride polymer, carbon fiber, expanded PTFE, meta-phenylenediamine and terephthaloyl chloride polymer, nylon, fiber glass, cotton, polypropylene and ceramic, rubber, latex, silicone, polyurethane, polyisoprene, polystyrene and polybutadiene polymer, urethane, polyethylene, polyisoprene, polyvinylchloride, ethylene propylene diene monomer, neoprene, and styrene butadiene.

9. The trocar of claim 7, wherein the instrument seal is coated in at least one compound selected from the group consisting of hydrophilic polymer coatings, Teflon, thermoplastic, cyanoacrylate, parylene, plasma surface treatments, cornstarch powder, silicone oil, silicone grease, astroglide lubricants, mineral oil, glycerin, alcohol, saline, Teflon lubricants, Krytox lubricants, molybdenum disulfide lubricants, and graphite.

10. The trocar of claim 7, wherein the instrument seal further comprises at least one of the following seals selected from the group consisting of a diaphragm, a septum, a flapper seal, a braid, and a duckbill valve.

11. The trocar of claim 8, wherein the instrument seal is a diaphragm, wherein the diaphragm is interacting and interlocking.

12. The trocar of claim 1, further comprising an instrument seal disposed on the proximal end of the distal assembly body, wherein the instrument seal is an air seal further comprising:
a pump;
at least two gas recirculation tubes fluidly connected to the interstitial space of the distal assembly body at one end and to the pump at an opposite end; and
at least one pressure barrier output jet positioned within the interstitial space of the distal assembly body and in fluid communication with the gas recirculation tubes;
wherein the pump supplies a continuous supply of gas to the at least one pressure barrier output jet through the gas recirculation tubes, thereby forming a pressure barrier with the gas expelled by the at least one pressure barrier output jet.

13. The trocar of claim 1, wherein the proximal assembly body can be rotated to adjust the orientation of endoscopic instrumentation from anterior-posterior to lateral-medial orientation.

14. The trocar of claim 1, wherein the multichannel divider is disposed longitudinally along the entire length of the proximal assembly body.

15. The trocar of claim 12, wherein one of the gas recirculation tubes is an input tube.

16. The trocar of claim 15, wherein one of the gas recirculation tubes is an output tube.
